(19) United States
(12) Patent Application Publication
(10) Pub. No.: US 2018/0036163 A1
(43) Pub. Date: Feb. 8, 2018

(54) WRAP COMBINED IN A URINE SHEATH

(71) Applicant: WEN-HUA WANG, TAINAN CITY (TW)

(72) Inventor: WEN-HUA WANG, TAINAN CITY (TW)

(21) Appl. No.: 15/638,232

(22) Filed: Jun. 29, 2017

(30) Foreign Application Priority Data
Aug. 3, 2016 (TW) 105211748

(51) Int. Cl. A61F 5/453; A61F 5/44

(52) U.S. Cl. CPC A61F 5/453; A61F 5/4408; A61F 5/451

(57) ABSTRACT

This invention relates to a wrap combined in a urine sheath. The wrap is a tubular elastic and impermeable rubbery sheath with one end secured to an opening segment of the urine sheath, and the other end is expanded by the opening segment of the urine sheath to automatically form a leaking-stop ring and a wrap opening in the center of the leaking-stop ring; the wrap opening is used to wrap and position a male penis, so that the catheterization space in the urine sheath is isolated from the outside, which prevents the urine from leaking out and gives the expansion-shrinkage margin coping with a male penis erection and non-erection state, avoiding too tight constraint to the penis, so as to form a wrap combined in a urine sheath with better anti-leakage and comfortable wrapping efficiency.

### CROSS-REFERENCE TO RELATED APPLICATION

This application claims priority to Taiwanese patent application No. 105211748, filed on Aug. 3, 2016, entitled “Wrap Combined in a Urine Sheath”, the content of which is hereby incorporated by reference in its entirety.

### FIELD OF THE INVENTION

The present invention relates to a wrap that is combined in a urine sheath, and especially refers to a wrap that can be used to assist a urine sheath to prevent urine from being leaked. The wrap can moderately wrap a male penis to isolate the catheterization space in the urine sheath from the outside, so as to prevent leakage and effectively enhance the anti-leakage and comfortably wrapping efficiency of the urine sheath.

### DESCRIPTION OF RELATED ART

For long-term bedridden male patients and those who lose self-control, the more troubled problem is to urinate. At present, methods of dealing with urination usually adopt diaper or urine collection device, but because the diaper is covered in the lower body, it is easy to produce hot and humid feeling, and in order to avoid skin lesions, urethral infection, one must always observe and replace the diapers, which causes caregivers and patients deep inconvenience and trouble. To this end, most male patients wear urine collection device. A urine collection device has a straight-shaped or cup-shaped urine sheath (2), (3) with a catheterization space, which is connected to a catheter (5), a urine bag (6), and a urinary catheter (7) with a control valve (8) and connected to the urine bag (6), as shown in FIG. 5. When in use, the opening of urine sheath (2), (3) covers the male penis, so that the glans can be placed in the catheterization space (23) of urine sheath (2), (3). By band wrapping and adhesion positioning, when urinating, urine can be collected into urine bag (6) through urine sheath (2), (3) and then catheter (5) in turn. As soon as the urine reaches a certain amount, the control valve (8) is opened, and the urine will be discharged through urinary catheter (7). However, urine sheath (2), (3) is tightened by wrapping and adhering the band, it is easy to generate gaps due to the patient's turning over action resulting in relaxation, that will let the urine leak out through the gaps, causing soaking pollution, odor spilling, and even making urine sheath (2), (3) fall off. Therefore, in order to avoid generating the gaps and urine sheath (2), (3) falling off, one must make the band more tightly wrapped and adhered before the penis has not yet congested erection, but when the penis erects, it makes the patient uncomfortable, even causes the patient unable to urinate, which has become a unsolved problem.

### SUMMARY OF THE INVENTION

In order to solve the above problems in using a urine sheath and its disadvantages, the present invention provides a wrap combined in a urine sheath, allowing users to easily wear and not easy to fall off, which not only can enhance the comfort and smooth urination, but also completely present the urine from leaking out and ensure sanitary safety.

The present invention provides a wrap combined in a urine sheath. The wrap is a tubular elastic and impermeable rubbery sheath with one end secured onto the opening segment of a urine sheath, and the other end is expanded by the opening segment of the urine sheath to automatically to form a leaking-stop ring and a wrap opening in the center of the leaking-stop ring, to construct a wrap combined in a urine sheath.

The present invention provides a wrap combined in a urine sheath, using the wrap opening to position and wrap a male penis, so that the catheterization space in the urine sheath is isolated from the outside. It can prevent the urine in the catheterization space from leaking out, and give the expansion-shrinkage margin coping with the penis erection or non-erection state, so as to avoid the wrap opening causing too tight constraint to the penis, effectively enhance anti-leakage and comfortable wrapping efficiency of the urine sheath.

The present invention provides a wrap combined in a urine sheath, wherein the wrap is made by one of the elastic materials selected from rubber, plastic, latex, and silicone.

### BRIEF DESCRIPTION OF THE DRAWINGS

FIG. 1 is a side view of a wrap and a straight-shaped urine sheath according to an embodiment of the present invention.

FIG. 2 is a side view of a wrap combined with a straight-shaped urine sheath according to an embodiment of the present invention.

FIG. 3 is a diagram showing the use of the combination of a wrap and a straight-shaped urine sheath according to an embodiment of the present invention.

FIG. 4 is a diagram showing the combination of a wrap and a cup-shaped urine sheath according to an embodiment of the present invention.

FIG. 5 is a structure diagram of a known urine collection device.

### DETAILED DESCRIPTION OF THE PREFERRED EMBODIMENTS

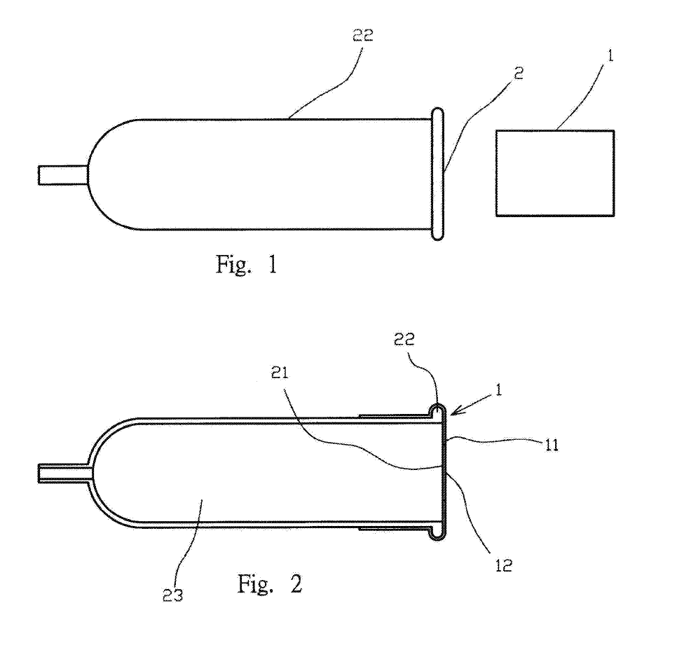

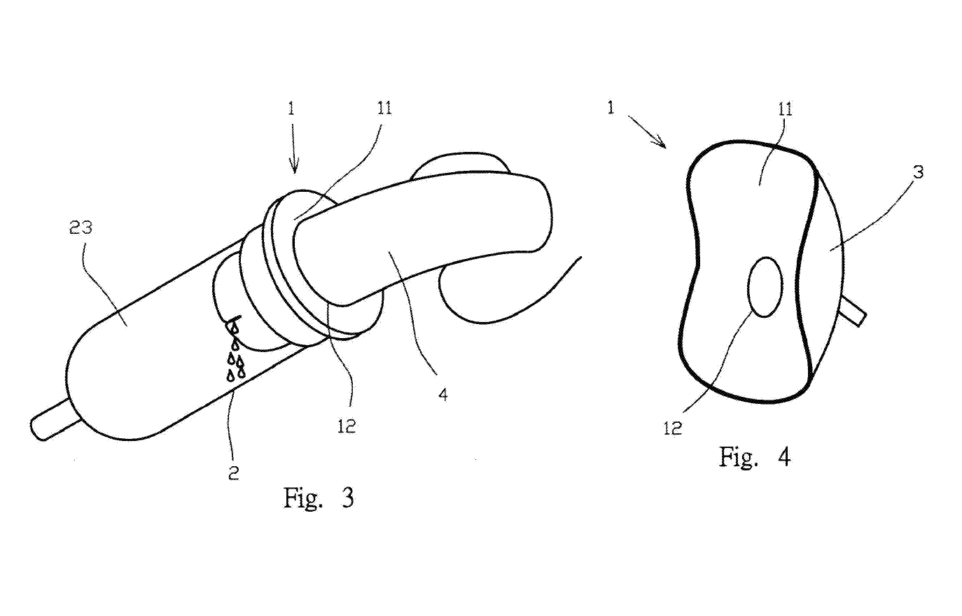

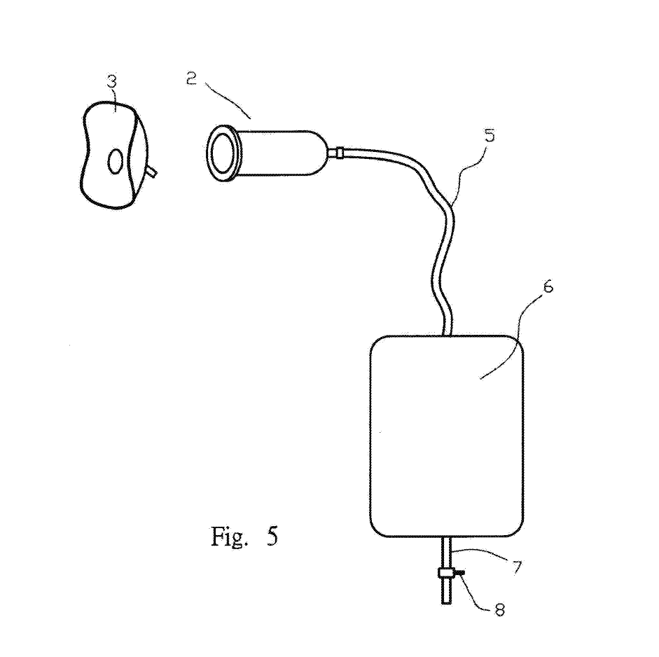

The present invention provides a wrap combined in a urine sheath. First referring to FIG. 1, FIG. 2 and FIG. 3, which show a preferred embodiment of present invention, the wrap (1) is a tubular elastic and impermeable rubbery sheath, made by one of the elastic materials selected from rubber, plastic, latex, and silicone, so as to have a better soft touch and expansion-shrinkage margin. One end of wrap (1) is secured onto an opening segment (21) of a straight-shaped urine sheath (2) via expansion and shrinkage elasticity, and the other end is expanded by the opening segment (21) of the urine sheath (2) to automatically form a leaking-stop ring (11), which can cover the opening (22) of the urine sheath, and a wrap opening (12) in the center of the leaking-stop ring (11). The wrap opening (12) can be used to position and wrap a male penis (4), so that the catheterization space (23) in the urine sheath (2) is isolated from the outside, preventing the urine in the catheterization space (23) from leaking out, and giving the expansion-shrinkage margin coping with the male penis (4) erection or non-erection state, so as to avoid the wrap opening (12) causing too tight constraint to the penis, which makes it very comfortable for the user to use, and can maintain the wrapping relationship with the penis (4) to avoid falling off.

Referring to FIG. 4, another preferred embodiment of the present invention is shown, which, as shown, is applicable to a general cup-shaped urine sheath (3). Otherwise, this embodiment structurally and in use is exactly same as the above straight-shaped urine sheath (2) embodiment, and the difference is only in the shape of the urine sheath, so repeated description will not be given here.

In summary, one end of wrap (1) of the present invention is secured onto the opening segment (21) of the urine sheath (2), the other end forms a leaking-stop ring (11) and a wrap opening (12), which can be positioned onto male penis (4), so that regardless of lying, turning over, standing or moving, due to the isolation of leaking-stop ring (11), when urinating, the urine will never leak out, which effectively prevents wet pollution, odor spilling and other health problems. And because the expansion-shrinkage margin of the wrap opening (12) can be moderately expanded or contracted at the penis erection or non-erection state, so as to avoid too tight constraint to the penis (4), and the wrap (1) can be used more comfortably and smooth urination is achieved.

The embodiments disclosed herein are for the purpose of illustrating the feature of the present invention and are not intended to limit the scope of the present invention. All the changes and modifications made according to the scope of the present invention are covered by the claims of the present invention.

What is claimed is:

1. A wrap combined in a urine sheath, the wrap being a tubular elastic and impermeable rubbery sheath with a soft touch and an expansion-shrinkage margin, wherein one end of the wrap is secured onto an opening segment of the urine sheath, and the other end of the wrap is expanded by an opening of the urine sheath to automatically form a leaking-stop ring, which covers the opening of the urine sheath, and a wrap opening in the center of the leaking-stop ring, which can wrap a male penis.

2. A wrap combined in a urine sheath according to claim 1, wherein the wrap is made by one of elastic materials selected from rubber, plastic, latex, and silicone.
